(19) United States
(12) Patent Application Publication
(10) Pub. No.: US 2020/0155300 A1
(43) Pub. Date: May 21, 2020

(54) AUTOMATIC HAIR IMPLANT APPARATUS

(71) Applicant: Electronics and Telecommunications Research Institute, Daejeon (KR)

(72) Inventors: Tae Wuk BAE, Daegu (KR); Kyu Hyung KIM, Daegu (KR); Jung Wook SUH, Daegu (KR); Eun Chang CHOI, Daegu (KR); Chang Hyuk HONG, Daegu (KR)

(73) Assignee: Electronics and Telecommunications Research Institute, Daejeon (KR)

(21) Appl. No.: 16/684,273

(22) Filed: Nov. 14, 2019

(30) Foreign Application Priority Data
Nov. 19, 2018 (KR) 10-2018-0142520
Mar. 6, 2019 (KR) 10-2019-0026004

(51) Int. Cl. A61F 2/10; A61F 2/00

(52) U.S. Cl. CPC A61F 2/10; A61F 2/0095

(57) ABSTRACT

An automatic hair implant apparatus according to the present disclosure includes a cylinder including a nozzle at an exit side and having a supporting part partially opened on a path, an implant unit configured to pass through the nozzle to implant an embedded follicle into target skin, a cartridge disposed on the supporting part of the cylinder and configured to sequentially replace the implant unit, and a push rod configured to guide the implant unit to the nozzle and return to its original position when the follicle is implanted into the target skin.

### CROSS-REFERENCE TO RELATED APPLICATION

This application claims priority to and the benefit of Korean Patent Application Nos. 10-2018-0142520 and 10-2019-0026004, filed on Nov. 19, 2018 and Mar. 6, 2019, the disclosure of which is incorporated herein by reference in its entirety.

### BACKGROUND

### 1. Field of the Invention

The present disclosure relates to an automatic hair implant apparatus, and more specifically, to an automatic hair implant apparatus capable of continuously implanting a follicle by automatically replacing an implant unit, in which the follicle is embedded, during a hair implant procedure.

### 2. Discussion of Related Art

Generally, a procedure of wearing a wig or implanting hair in a hair loss portion for cosmetic purposes to conceal the hair loss portion is increasing.

A manual follicle implanting device is used in the procedure process. The manual follicle implanting device inserts a needle in which a follicle is accommodated into the skin and then presses the follicle inserted into the skin using a bar in a process of retracting the needle. Accordingly, the follicle can be intactly implanted into the skin without escaping to the outside.

In the case of a manual follicle implanting device which is currently used, since roughly 2,000 follicles (graft) are involved in a single procedure on one patient, patient and doctor fatigue can increase. Further, a procedure has no choice but to be long, and thus costs for the above are expensive and commercialization is limited.

A prior art document related to the present disclosure is Korean Patent Registration No. 10-0320880 (filed on Jan. 3, 2002), and a technology related to a hair implant apparatus is disclosed in the prior art document.

### SUMMARY OF THE INVENTION

The present disclosure is directed to an automatic hair implant apparatus capable of continuously implanting a follicle by automatically replacing an implant unit, in which a hair follicle is embedded, during an implant procedure.

Technical problems desired to be solved by the present disclosure are not limited to the above-described problems, and other technical problems which are not mentioned can be apparently understood by those skilled in the art from the specification and the accompanying drawings.

According to an aspect of the present disclosure, there is provided an automatic hair implant apparatus including: a cylinder including a nozzle at an exit side and having a supporting part partially opened on a path; an implant unit configured to pass through the nozzle to implant an embedded follicle into target skin; a cartridge disposed on the supporting part of the cylinder and configured to sequentially replace the implant unit; and a push rod configured to guide the implant unit to the nozzle and return to its original position when the follicle is implanted into the target skin.

In this case, the automatic hair implant apparatus may further include a driving part configured to drive and control the cartridge and the push rod.

Here, the driving part may reciprocally move the cartridge in a width direction of the cylinder and reciprocally move the push rod in a longitudinal direction of the cylinder, the implant unit and the push rod may be collinearly disposed whenever the cartridge moves at a predetermined interval, and the push rod may be returned to its original position when the push rod guides the implant unit to the nozzle and thus the follicle embedded in the implant unit is implanted into the target skin for a predetermined time.

The driving part may include: a first location adjusting unit configured to reciprocally move the cartridge in the width direction of the cylinder on the supporting part; a second location adjusting unit configured to reciprocally move the push rod in the longitudinal direction of the cylinder; and a controller configured to control the first location adjusting unit and the second location adjusting unit.

The controller may be provided so that the implant unit and the push rod are collinearly disposed whenever the cartridge moves at the predetermined interval through the first location adjusting unit, and the push rod is returned to its original position when the push rod guides the implant unit to the nozzle through the second location adjusting unit and thus the follicle embedded in the implant unit is implanted into the target skin for the predetermined time.

The implant unit may include: a housing; a hollow needle configured to protrude in a longitudinal direction of the housing and having a slit in which the follicle is embedded; and a buffering member configured to surround the needle.

The needle may be formed in a structure which is detachably attached to the housing.

In this case, the implant unit may include a connection member configured to attach and detach the needle to and from the housing.

The needle may include an exit path which is open in one direction and formed in an inclined shape at one end portion thereof, and the housing may include a direction protrusion formed to protrude to an outer side to have a direction the same as that of the exit path.

The cartridge may be formed in a plate shape and move in a serial manner.

The cartridge may be formed in a cylindrical shape and move in a circular motion.

The cylinder may include a step protrusion configured to partially protrude toward an inner side, and the push rod may be attached to and detached from the step protrusion by a rotating operation.

According to another aspect of the present disclosure, there is provided an automatic hair implant apparatus including: a cylinder configured to form a channel guiding an implanting path of a follicle, including a nozzle at an exit side where the follicle is implanted, and having a supporting part partially opened on a path; an implant unit including a housing, a hollow needle configured to protrude in a longitudinal direction of the housing and having a slit in which the follicle is embedded, and a buffering member configured to surround the needle; a cartridge disposed on the supporting part of the cylinder to accommodate a plurality of implant units, and configured to sequentially locate the implant unit on the implanting path of the follicle so that the needle faces the nozzle; a push rod configured to guide the implant unit to the nozzle so that the needle passes through the nozzle and thus the follicle is implanted into target skin; and a driving part configured to drive and control the cartridge and the push rod.

The driving part may set the implant unit and the push rod so that the implant unit and the push rod may be collinearly disposed whenever the cartridge is moved.

The driving part may return the push rod to its original position when the push rod guides the implant unit to the nozzle and thus the follicle is implanted into the target skin.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1 is a perspective view illustrating an automatic hair implant apparatus according to a first embodiment of the present disclosure;

FIG. 2 is a view illustrating an operational relationship of the automatic hair implant apparatus according to the first embodiment of the present disclosure;

FIGS. 3A and 3B are views illustrating an implant unit of the automatic hair implant apparatus according to the first embodiment of the present disclosure;

FIG. 4 is a view illustrating an automatic hair implant apparatus according to a second embodiment of the present disclosure;

FIG. 5 is a view illustrating an operational relationship of the automatic hair implant apparatus according to the second embodiment of the present disclosure;

FIG. 6 is a view illustrating an operation structure of the automatic hair implant apparatus according to the second embodiment of the present disclosure;

FIGS. 7A to 7H are views illustrating an operation state of the automatic hair implant apparatus according to the second embodiment of the present disclosure; and

FIGS. 8A to 8H are cross-sectional views illustrating the operation state of the automatic hair implant apparatus according to the second embodiment of the present disclosure.

### DETAILED DESCRIPTION OF EXEMPLARY EMBODIMENTS

Advantages and characteristics of the present disclosure, and a method of achieving the above, will be apparent with reference to embodiments which will be described in detail with the accompanying drawings. However, the present disclosure is not limited to the embodiments which will be described below and may be implemented in different forms. The embodiments are only provided to completely disclose the present disclosure and completely convey the scope of the present disclosure to those skilled in the art, and the present disclosure is defined by the disclosed claims. Meanwhile, terms used in the description are provided not to limit the present disclosure but to describe the embodiments. In the embodiment, the singular form is intended to also include the plural form unless the context clearly indicates otherwise. The terms “comprise” and/or “comprising” as used herein do not preclude the presence or addition of at least one other component, step, operation, and/or element other than the stated components, steps, operations and/or elements.

Hereinafter, preferable embodiments of the present disclosure will be described in detail with reference to the accompanying drawings.

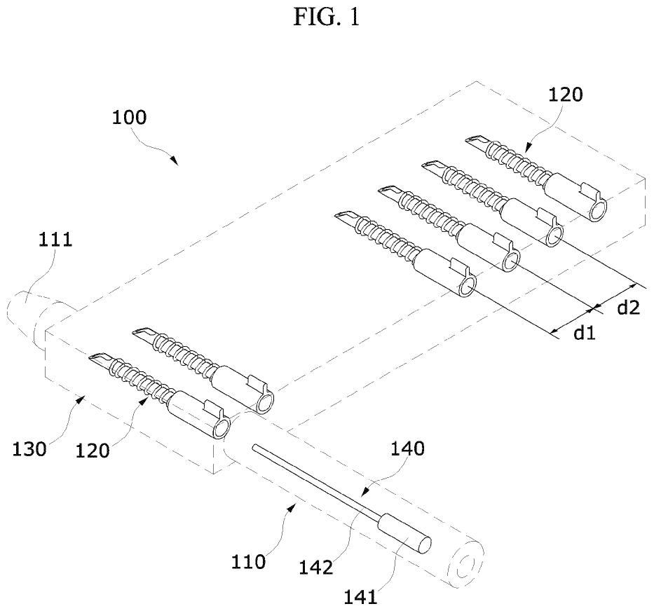

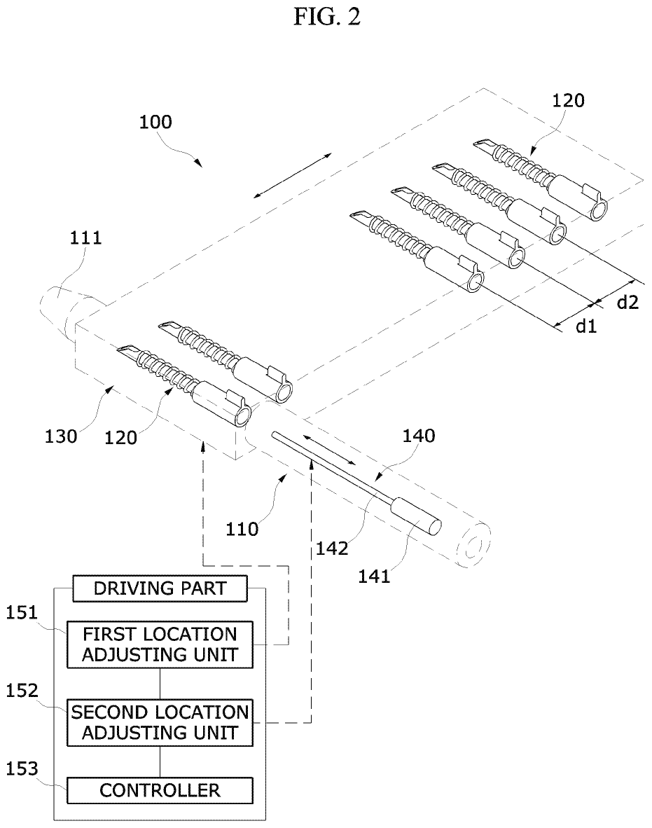

FIG. 1 is a perspective view illustrating an automatic hair implant apparatus according to a first embodiment of the present disclosure, and FIG. 2 is a view illustrating an operational relationship of the automatic hair implant apparatus according to the first embodiment of the present disclosure.

Referring to both FIGS. 1 and 2, an automatic hair implant apparatus 100 may roughly include a cylinder 110, implant units 120, a cartridge 130, a push rod 140, and a driving part 150.

The cylinder 110 includes a nozzle 111 at an exit side, and has a supporting part, which is partially open, on a path. The cylinder 110 provides an implanting path through which a follicle is implanted.

The implant unit 120 serves to implant the follicle, embedded therein by passing through the nozzle 111 provided in the cylinder 110, into target skin. Here, the target skin refers to skin into which the follicle is implanted, and corresponds to all skin types into which follicles may be inserted.

The cartridge 130 has a structure capable of accommodating a plurality of implant units 120. The cartridge 130 may move in a width direction (a lateral direction) of the cylinder 110. In this case, the cartridge 130 is disposed on the supporting part of the cylinder 110, and sequentially replaces the implant units 120.

The push rod 140 serves to guide the implant unit 120 to the nozzle 111 and return to its original position when the follicle is implanted into the target skin.

The driving part 150 drives and controls the cartridge 130 and the push rod 140. In this case, the driving part 150 may reciprocally move the cartridge 130 in the width direction of the cylinder 110, and reciprocally move the push rod 140 in a longitudinal direction (a vertical direction) of the cylinder 110.

Further, the driving part 150 collinearly disposes the implant unit 120 and the push rod 140 whenever the cartridge 130 moves at a predetermined interval. Here, the predetermined interval refers to distances d1 and d2 between the plurality of implant units 120.

The driving part 150 may return the push rod 140 to its original position when the push rod 140 guides the implant unit 120 to the nozzle 111 and thus the follicle embedded in the implant unit 120 is implanted into the target skin for a predetermined time.

Here, the predetermined time refers to the time taken for the implant unit 120 to implant the follicle into the skin, the implant unit 120 to immediately return to its original position, and the push rod 140 to hold the follicle so that the follicle is not separated with the implant unit 120. Accordingly, the predetermined time may be changed according to a procedure environment.

The driving part 150 may move the cartridge 130 and the push rod 140 with one motor.

The driving part 150 may have a configuration including a first location adjusting unit 151, a second location adjusting unit 152, and a controller 153.

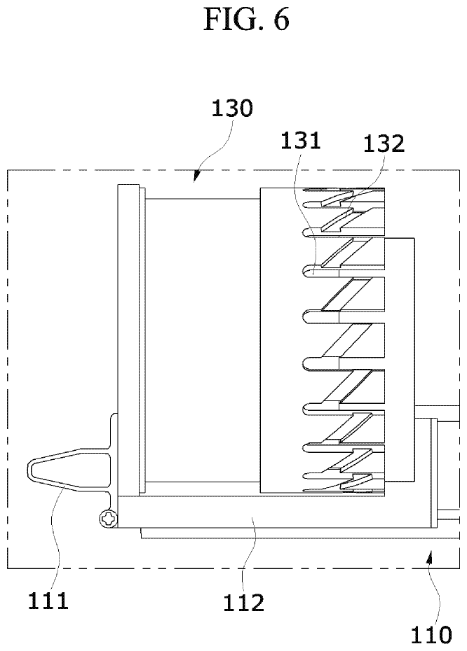

The first location adjusting unit 151 reciprocally moves the cartridge 130 in the width direction of the cylinder 110 on a supporting part (112 in FIG. 6) provided in the cylinder 110.

The second location adjusting unit 152 reciprocally moves the push rod 140 in the longitudinal direction of the cylinder 110.

The controller 153 controls the first and second location adjusting units 151 and 152. In this case, the controller 153 collinearly disposes the implant unit 120 and the push rod 140 whenever the cartridge 130 moves at the predetermined interval by controlling the first location adjusting unit 151.

In this case, the controller 153 controls the second location adjusting unit 152, and returns the push rod to its original position when the push rod 140 guides the implant unit 120 to the nozzle 111 through the second location adjusting unit 152 and thus the follicle embedded in the implant unit 120 is implanted into the targeted skin for the predetermined time.

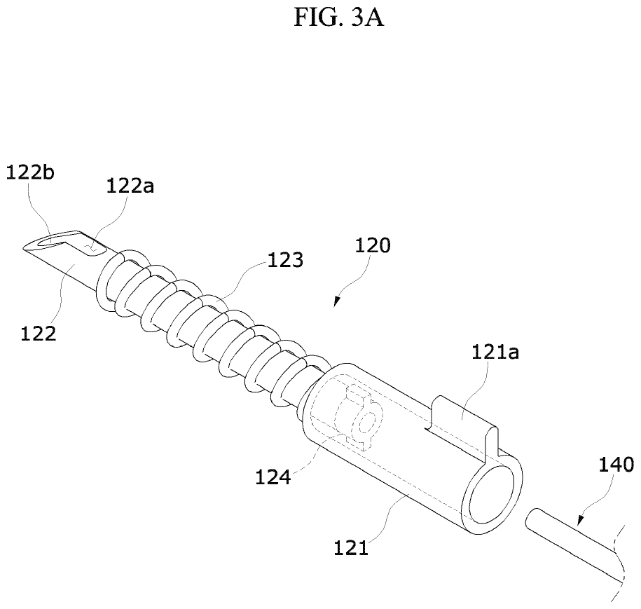

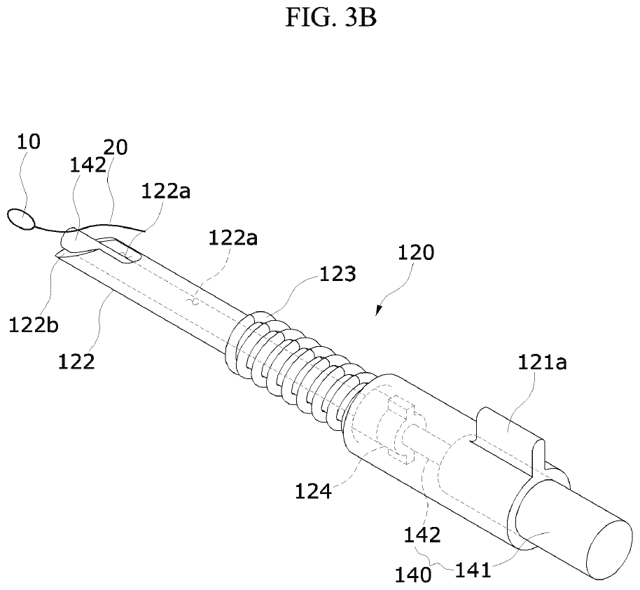

FIGS. 3A and 3B views illustrating an implant unit of the automatic hair implant apparatus according to the first embodiment of the present disclosure.

Referring to both FIGS. 3A and 3B, the implant unit 120 includes a housing 121, a needle 122, and a buffering member 123.

The housing 121 forms a body of the implant unit 120.

The needle 122 is a hollow needle, and protrudes in a longitudinal direction of the housing 121. In this case, the needle 122 has a slit 122a in which a follicle 10 is embedded.

The slit 122a is formed through a longitudinal direction of the needle 122. The needle 122 includes an exit path 122b which is open in one direction and formed in an inclined shape at one end portion thereof.

In this case, the housing 121 includes a direction protrusion 121a formed to protrude toward an outer side to have a direction the same as that of the exit path 122b.

The direction protrusion 121a is a structure configured to determine a direction of the slit 122a of the needle 122, and the direction of the slit 122a may be confirmed by the direction protrusion 121a.

The needle 122 is integrally formed with the housing 121.

In some cases, the needle 122 may be formed in a structure detachably attached to the housing 121. Accordingly, the implant unit 120 may include a connection member 124 configured to detachably attach the needle 122 to the housing 121.

The connection member 124 may be detachably attached to the inside of the housing 121 in the form of a separate screw thread (not shown) or concavo-convex coupling.

As shown in FIGS. 3A and 3B, the push rod 140 may be located collinearly with the slit 122a of the needle 122 to move. The push rod 140 includes a body 141 and a rod member 142.

The body 141 forms an exterior of the push rod 140.

The rod member 142 is formed in a bar shape which protrudes in a longitudinal direction from the body 141. The rod member 142 passes through the slit 122a of the needle 122 and serves to fix the follicle 10 in a state in which the follicle 10 is inserted into the skin targeted by the needle 122. At this time, the needle 122 returns to its original position due to the buffering member 123.

The above form provides convenience to an operator which actually inserts the follicle 10. In other words, since the above form is formed as a structure in which the follicle 10 is inserted into and retracts from the target skin automatically, an injury to the follicle 10 may be minimized.

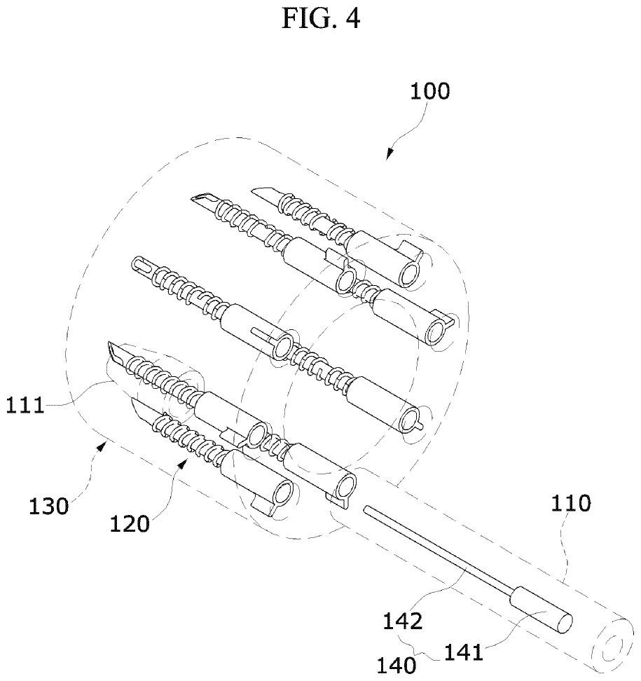

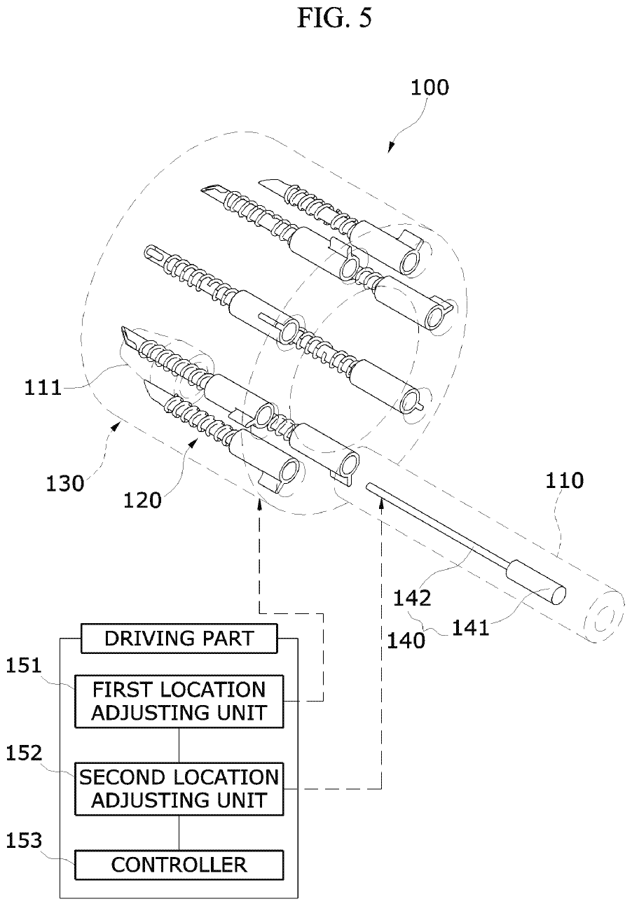

FIG. 4 is a view illustrating an automatic hair implant apparatus according to a second embodiment of the present disclosure, and FIG. 5 is a view illustrating an operational relationship of the automatic hair implant apparatus according to the second embodiment of the present disclosure.

Referring to both FIGS. 4 and 5, an automatic hair implant apparatus 100 may include a cylinder 110, implant units 120, a cartridge 130, a push rod 140, and a driving part 150.

Here, descriptions of parts similar to those of the automatic hair implant apparatus 100 according to the above-described first embodiment will be omitted.

The cartridge 130 may be formed in a cylindrical shape and may move in a circular motion. In this case, the cartridge 130 may be formed in a hollow shape in a radial manner. The implant units 120 may be accommodated in the cartridge 130 in a radial manner. The implant units 120 are accommodated in the cartridge 130 at a predetermined interval.

The cartridge 130 may be formed in a structure which is rotatable on a supporting part (112 in FIG. 6) of the cylinder 110.

A first location adjusting unit 151 may move the cartridge 130 at a predetermined time interval in consideration of a time in which a follicle is implanted. The first location adjusting unit 151 and a second location adjusting unit 152 may be linked with each other so as to have one driving force.

FIG. 6 is a view illustrating an operation structure of the automatic hair implant apparatus according to the second embodiment of the present disclosure.

Referring to FIG. 6, the cartridge 130 has a cylindrical rotating structure. In this case, in a side surface of the cartridge 130, forward grooves 131 are provided in a horizontal direction, and diagonal grooves 132 are provided in a vertical direction.

In this case, the diagonal groove 132 has a predetermined step with the forward groove 131. That is, the diagonal groove 132 is formed to have a height smaller than that of the forward groove 131. That is, the implant unit (120 in FIG. 5) may be sequentially moved by one space at a time by rotating the cartridge 130 by one space at a time. Accordingly, the implant unit (120 in FIG. 5) and the push rod (140 in FIG. 5) may be collinearly disposed.

In a rotating method of the cartridge 130, since a separate moving shaft (not shown) provided in the first location adjusting unit (151 in FIG. 5) moves the forward groove 131 and the diagonal groove 132, the cartridge 130 moves along a rotating radius, and the implant unit (120 in FIG. 5) may move frontward and backward.

The cartridge 130 is connected to an upper portion of the supporting part 112 disposed on a path of the cylinder 110. The supporting part 112 has a rotatable connection structure with the cartridge 130.

The cartridge 130 may be directly connected to a nozzle 111 to minimize a moving distance of a needle (122 in FIG. 5).

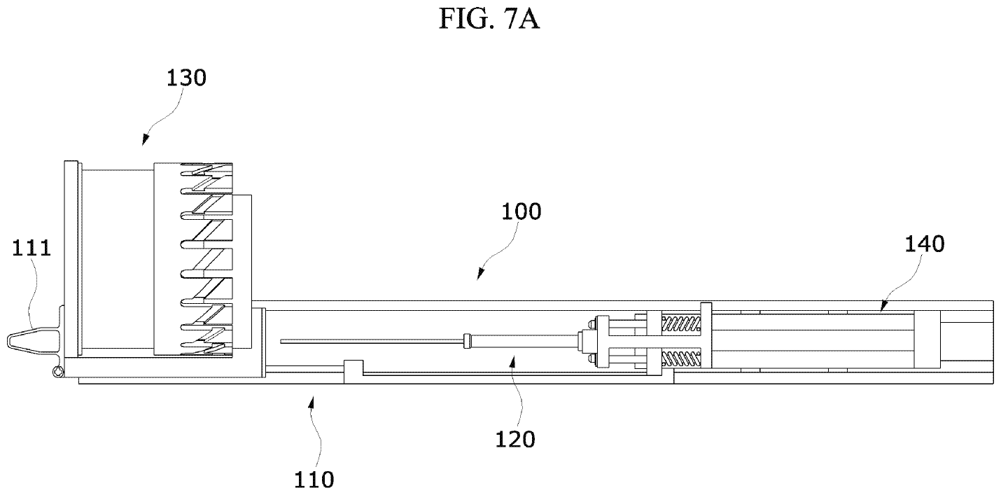

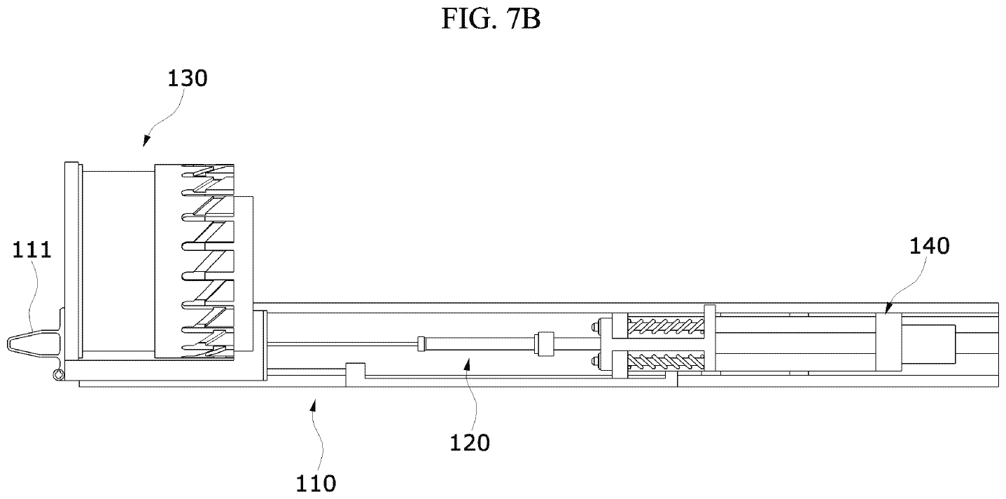

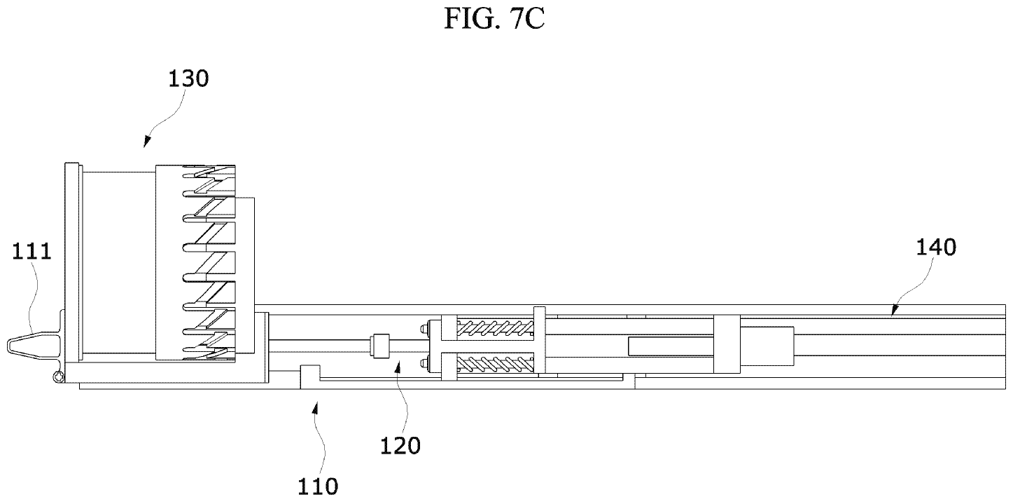

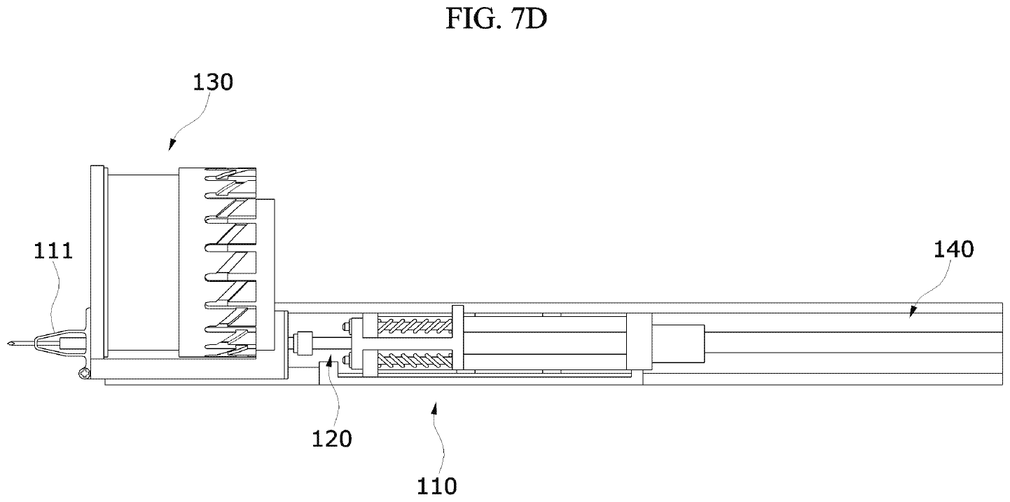

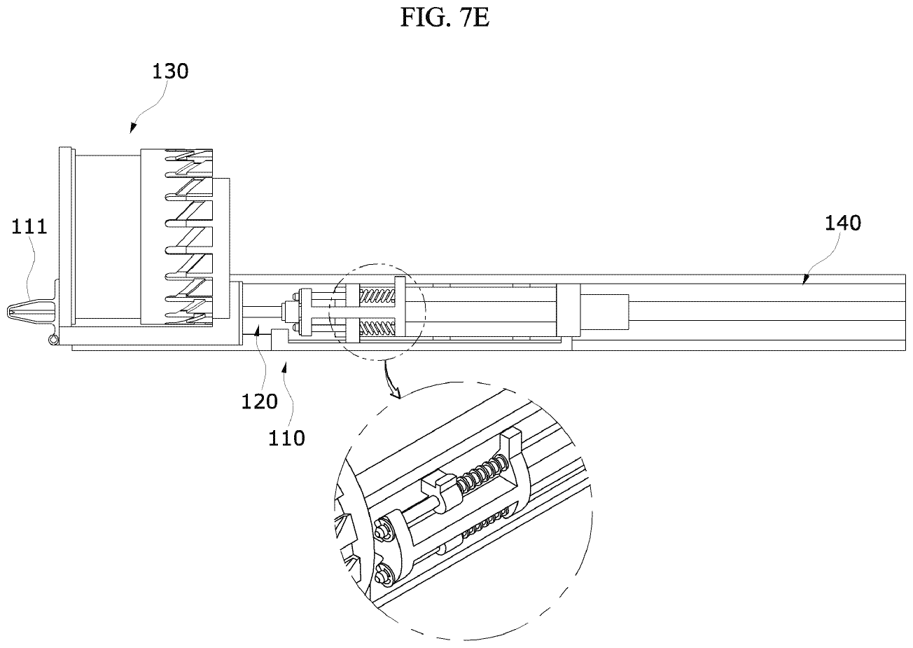

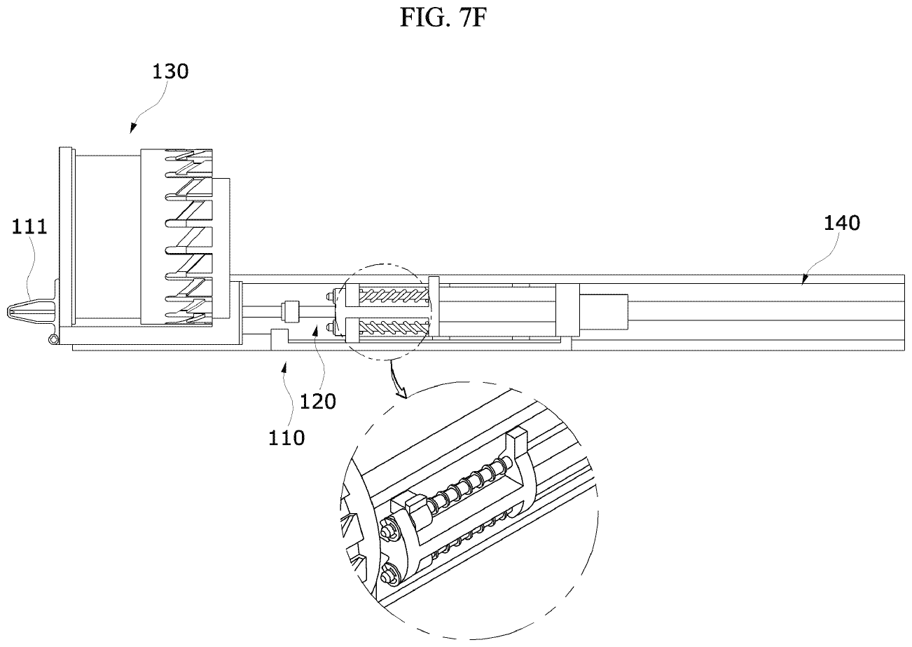

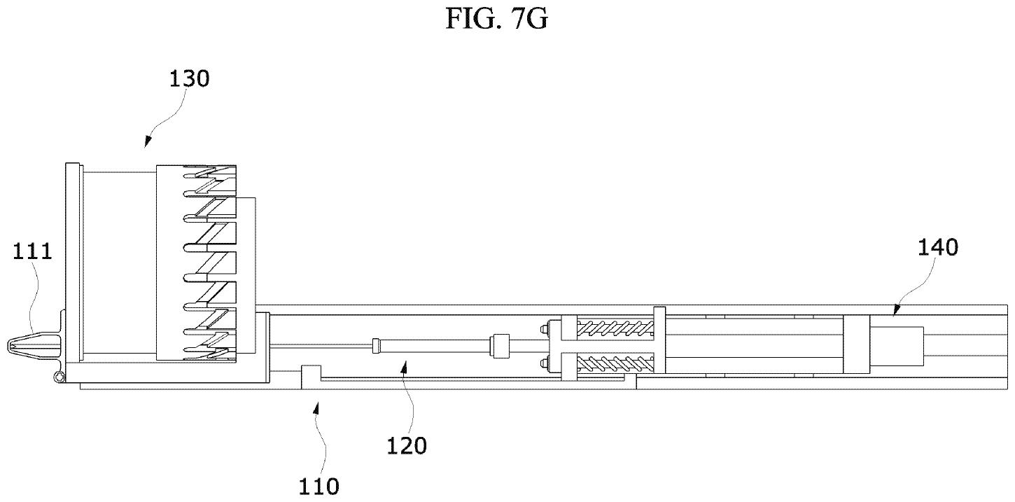

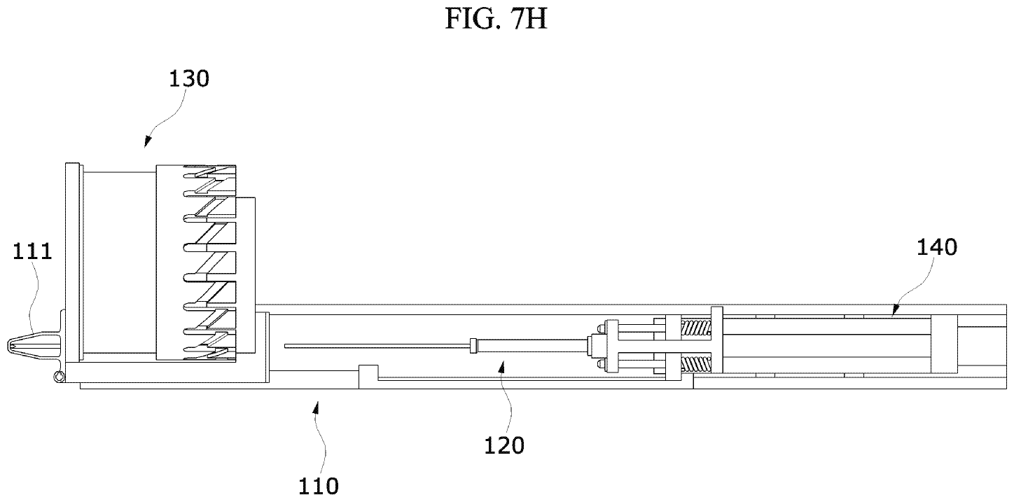

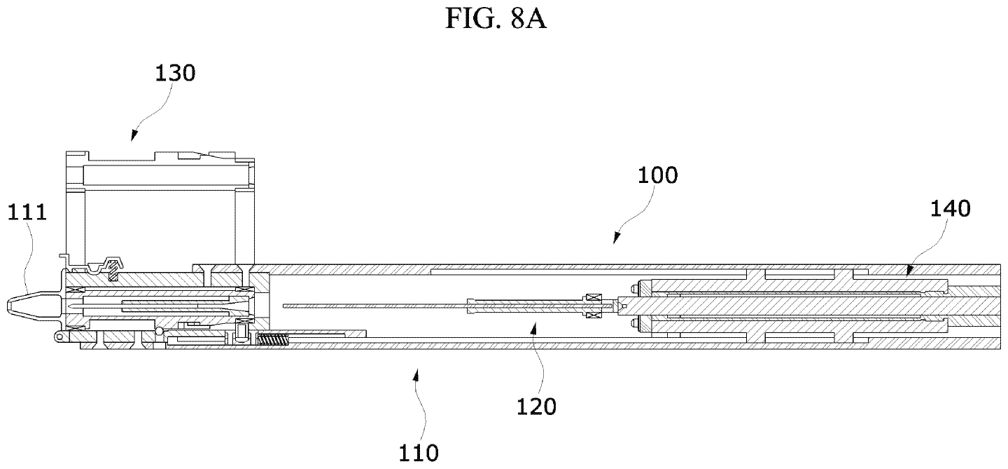

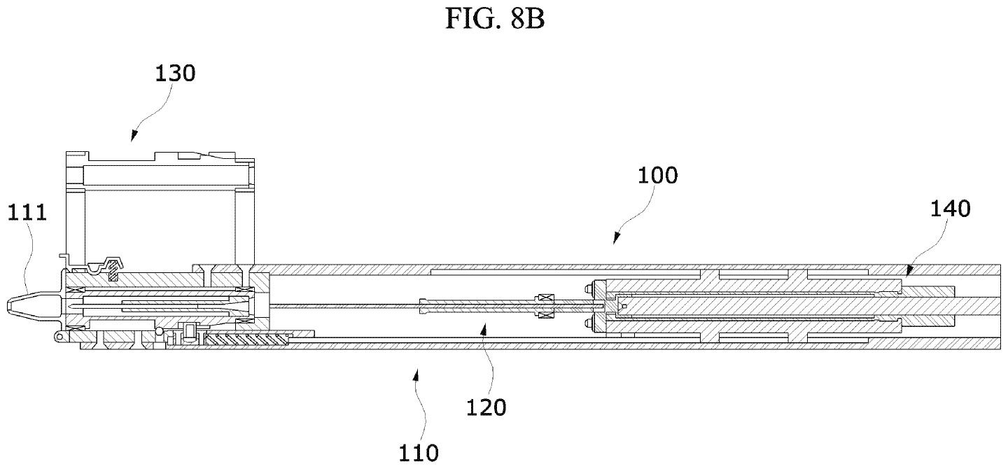

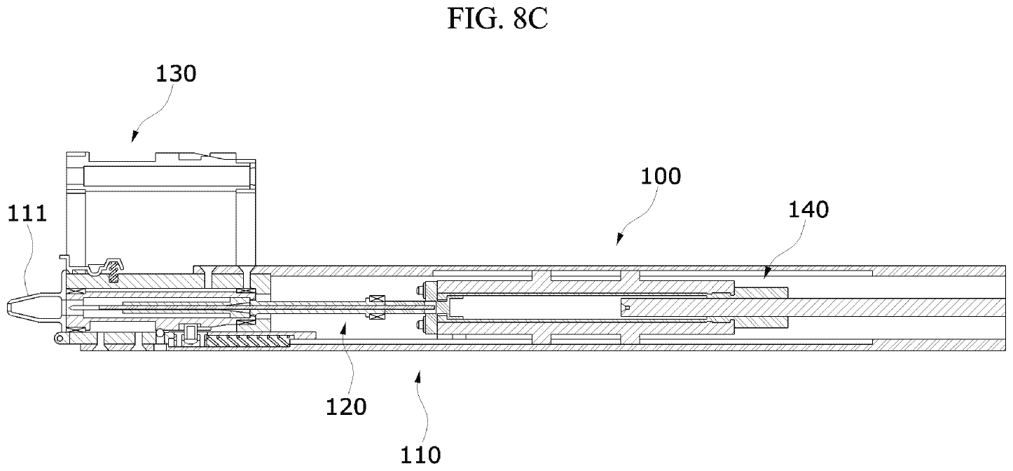

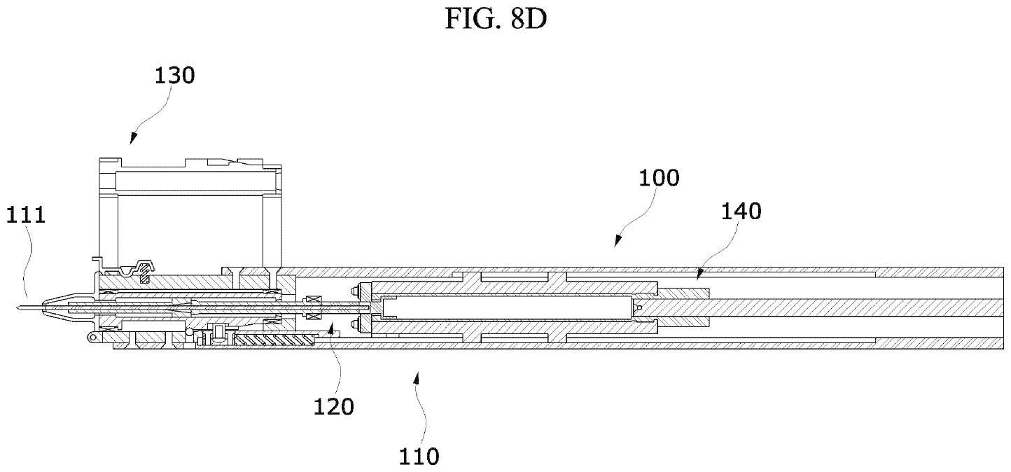

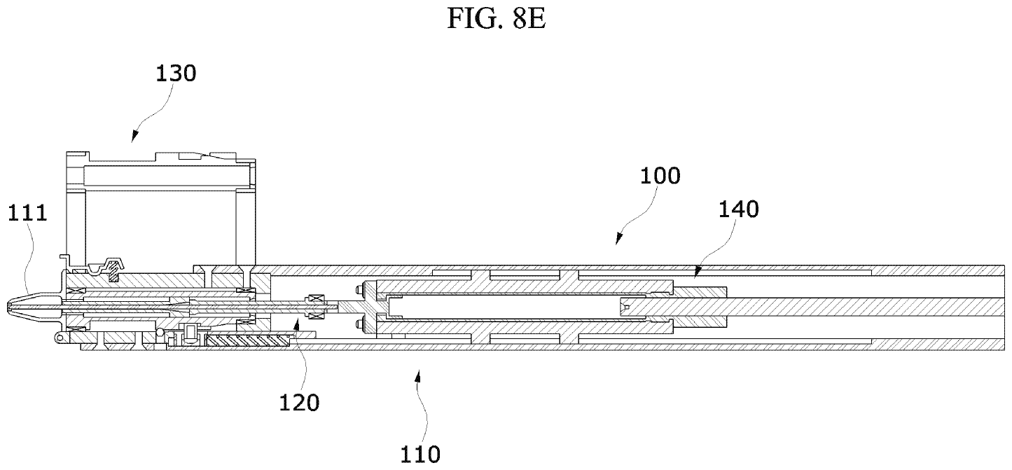

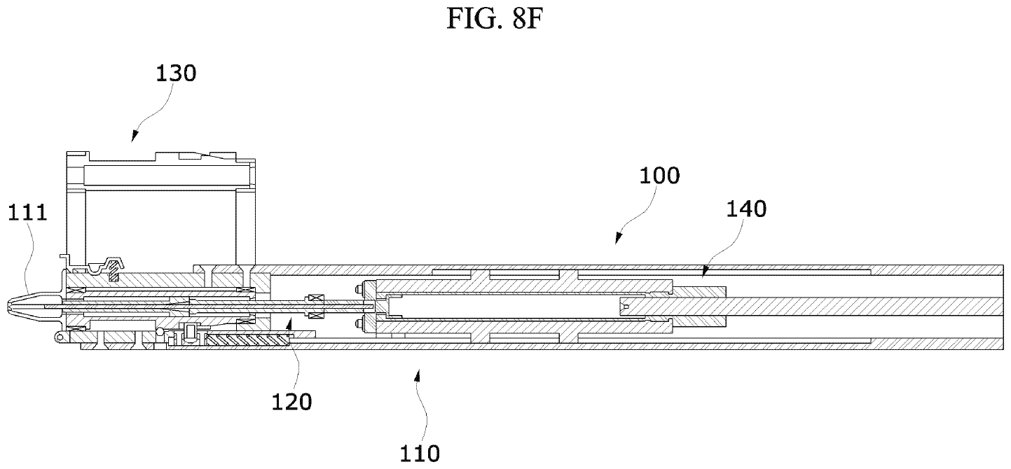

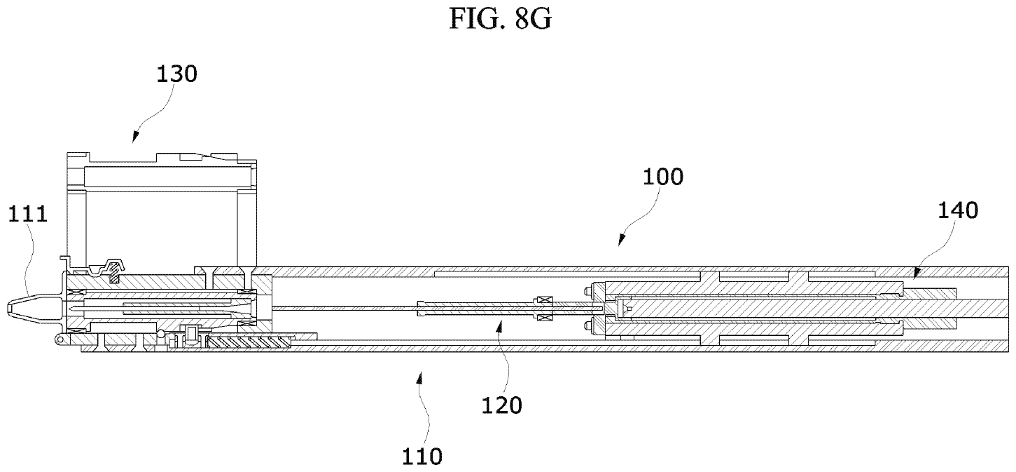

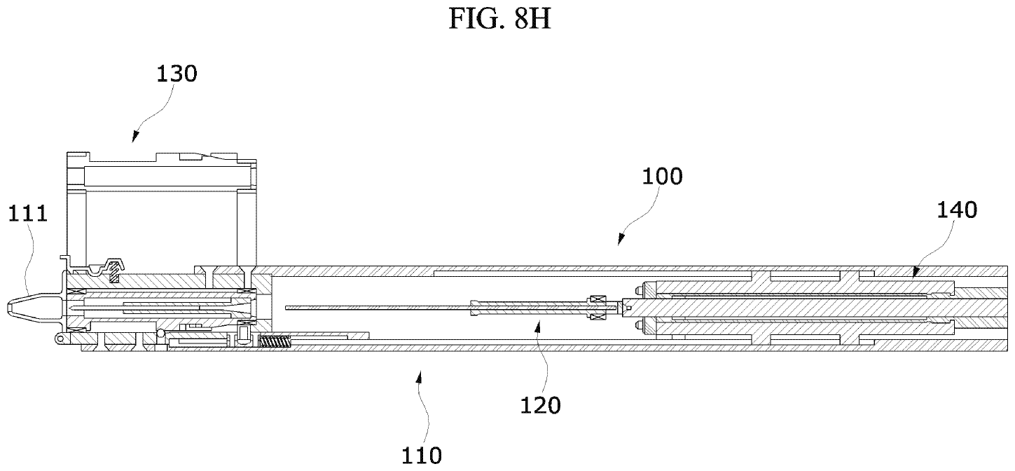

FIGS. 7A to 7H are views illustrating an operation state of the automatic hair implant apparatus according to the second embodiment of the present disclosure, and FIGS. 8A to 8H are cross-sectional views illustrating the operation state of the automatic hair implant apparatus according to the second embodiment of the present disclosure.

Referring to both FIGS. 7A to 7H and 8A to 8H, in FIGS. 7A and 8A, the push rod 140 is located at the outside of the cartridge 130 at a starting position.

In FIGS. 7B and 8B, the cartridge 130 is located at a regular position to which the implant unit 120 is matched. At this time, the implant unit 120 moves to an inlet of the cartridge 130.

In FIGS. 7C and 8C, the push rod 140 is inserted into the cartridge 130.

In FIGS. 7D and 8D, the moving shaft (not shown) provided in the first location adjusting unit is advanced to push the implant unit 120. At this time, the push rod 140 stops to seat the follicle embedded in the implant unit 120 into the scalp. Further, the implant unit 120 is retracted.

In FIGS. 7E and 8E, the push rod 140 is fixed by a step protrusion (not shown) provided in the cylinder 110. At this time, only the implant unit 120 is retracted. The push rod 140 pushes the follicle so that the follicle may be seated into the scalp.

In FIGS. 7F and 8F, when a predetermined time passes, the push rod 140 returns to its original position. At this time, since a separate press member (not shown) provided in the first location adjusting unit pushes the step protrusion, fixing of the push rod 140 is released. Accordingly, the push rod 140 returns (retracts) to its original position.

In FIGS. 7F, 7G 7H, 8F, 8G and 8H, the implant unit 120 and the push rod 140 simultaneously return to their original positions.

As described above, the automatic hair implant apparatus which is the present disclosure may automatically replace the implant unit in which the follicle is embedded during a hair implant procedure.

Accordingly, operator fatigue may be reduced and efficiency of the procedure may be improved by allowing the follicle to be continuously implanted.

Further, a procedure time may be effectively reduced.

An automatic hair implant apparatus according to an embodiment of the present disclosure can automatically replace an implant unit in which a follicle is embedded during a hair implant procedure.

Accordingly, operator fatigue can be reduced and efficiency of the procedure can be improved by allowing the follicle to be continuously implanted.

Further, a procedure time can be effectively reduced.

The present disclosure is not limited to the above-described embodiments and may be variously modified within the scope of the technical spirit of the present disclosure.

What is claimed is:

1. An automatic hair implant apparatus comprising:
a cylinder including a nozzle at an exit side, and having a supporting part partially opened on a path;
an implant unit configured to pass through the nozzle to implant an embedded follicle into target skin;
a cartridge disposed on the supporting part of the cylinder, and configured to sequentially replace the implant unit; and
a push rod configured to guide the implant unit to the nozzle and return to its original position when the follicle is implanted into the target skin.

2. The automatic hair implant apparatus of claim 1, further comprising a driving part configured to drive and control the cartridge and the push rod.

3. The automatic hair implant apparatus of claim 2, wherein:
the driving part reciprocally moves the cartridge in a width direction of the cylinder, and reciprocally moves the push rod in a longitudinal direction of the cylinder;
the implant unit and the push rod are collinearly disposed whenever the cartridge moves at a predetermined interval; and
the push rod is returned to its original position when the push rod guides the implant unit to the nozzle and thus the follicle embedded in the implant unit is implanted into the target skin for a predetermined time.

4. The automatic hair implant apparatus of claim 2, wherein the driving part includes a first location adjusting unit configured to reciprocally move the cartridge in a width direction of the cylinder on the supporting part; a second location adjusting unit configured to reciprocally move the push rod in a longitudinal direction of the cylinder; and a controller configured to control the first location adjusting unit and the second location adjusting unit.

5. The automatic hair implant apparatus of claim 4, wherein the controller is provided so that the implant unit and the push rod are collinearly disposed whenever the cartridge moves at the predetermined interval through the first location adjusting unit, and the push rod is returned to its original position when the push rod guides the implant unit to the nozzle through the second location adjusting unit and thus the follicle embedded in the implant unit is implanted into the target skin for the predetermined time.

6. The automatic hair implant apparatus of claim 1, wherein the implant unit includes a housing; a hollow needle configured to protrude in a longitudinal direction of the housing and having a slit in which the follicle is embedded; and a buffering member configured to surround the needle.

7. The automatic hair implant apparatus of claim 6, wherein the needle is formed in a structure detachable from the housing.

8. The automatic hair implant apparatus of claim 6, wherein the implant unit includes a connection member configured to attach and detach the needle to and from the housing.

9. The automatic hair implant apparatus of claim 6, wherein:
the needle includes an exit path which is open in one direction and formed in an inclined shape at one end portion thereof; and
the housing includes a direction protrusion formed to protrude to an outer side to have a direction the same as that of the exit path.

10. The automatic hair implant apparatus of claim 1, wherein the cartridge is formed in a plate shape and moves in a serial manner.

11. The automatic hair implant apparatus of claim 1, wherein the cartridge is formed in a cylindrical shape and moves in a circular motion.

12. The automatic hair implant apparatus of claim 1, wherein:
the cylinder includes a step protrusion configured to partially protrude toward an inner side; and
the push rod is attached to and detached from the step protrusion by a rotating operation.

13. An automatic hair implant apparatus comprising:
a cylinder configured to form a channel guiding an implanting path of a follicle, including a nozzle at an exit side where the follicle is implanted, and having a supporting part partially opened on a path;
an implant unit including a housing, a hollow needle configured to protrude in a longitudinal direction of the housing and having a slit in which the follicle is embedded, and a buffering member configured to surround the needle;
a cartridge disposed on the supporting part of the cylinder to accommodate a plurality of implant units, and configured to sequentially locate the implant unit on the implanting path of the follicle so that the needle faces the nozzle;
a push rod configured to guide the implant unit to the nozzle so that the needle passes through the nozzle and thus the follicle is implanted into target skin; and
a driving part configured to drive and control the cartridge and the push rod.

14. The automatic hair implant apparatus of claim 13, wherein the driving part sets the implant unit and the push rod so that the implant unit and the push rod are collinearly disposed whenever the cartridge is moved.

15. The automatic hair implant apparatus of claim 13, wherein the driving part returns the push rod to its original position when the push rod guides the implant unit to the nozzle and thus the follicle is implanted into the target skin.
